(19) United States
(12) Patent Application Publication
(10) Pub. No.: US 2022/0221419 A1
(43) Pub. Date: Jul. 14, 2022

(54) A BIOSENSOR FOR DETECTING AND CHARACTERIZING A BIOLOGICAL MATERIAL

(71) Applicant: UNIVERSITI MALAYA, kuala Lumpar (MY)

(72) Inventor: Vengadesh PERIASAMY, Kuala Lumpur (MY)

(21) Appl. No.: 17/615,044

(22) PCT Filed: Oct. 5, 2020
(86) PCT No.: PCT/MY2020/050097

§ 371 Date: Nov. 29, 2021

(30) Foreign Application Priority Data
Mar. 26, 2020 (MY) PI2020001608

(51) Int. Cl. G01N 27/327; G01N 33/487; G01N 27/417; G01N 27/416

(52) U.S. Cl. CPC G01N 27/3275; G01N 33/48707; G01N 27/4148; G01N 27/4163; G01N 27/417

(57) ABSTRACT

The present invention relates to a biosensor for detecting and characterising a biological material. The biosensor detects and characterises the biological material based on its electronic properties. The biosensor comprises at least two electrodes disposed on a substrate, a supply module, a frequency controller and a measurement module. The supply module, frequency controller and measurement module are connected to the at least two electrodes.

### FIELD OF INVENTION

The present invention relates to a biosensor for detecting and characterizing a biological material. More particularly, the present invention relates to a biosensor for detecting and characterizing a biological material based on an intrinsic signal specific to a particular biological material.

### BACKGROUND OF THE INVENTION

Detection of a biological material such as protein, nucleic acid, bacteria, virus, fungus, and many others in a sample is essential for application in medicine, disease diagnostic, vaccine development and various biological studies. Conventional procedures rely on the process of reacting a specific target biological material with a redox probe to generate chemical or physical reactions to perform identification and characterisation. These methods typically require complex laboratory setup and trained personnel to perform the operation and analysis.

In view of this, there is a need to provide a device and method to facilitate the detection of the biological material. An example of the device and method is disclosed in United States Patent Publication No. 2016/0146754 A1 which relates to a biosensor for detection and characterisation of analytes using electron-ionic mechanisms at fluid-sensor interfaces. The biosensor includes a semiconductor sensing element, a first electrode and a second electrode located on a first plane of the sensing element with a first electric field being applied thereacross, a third electrode located on a second plane of the sensing element parallel to and removed from the first plane with a second electric field being applied across the first electrode and the third electrode perpendicular to the first electric field, and a dielectric substrate having a first portion that constrains a fluid including an analyte on a surface of the sensing element, and a second portion that facilitates dielectric separation of the fluid from the electrodes. The mutually perpendicular electric fields facilitate adjusting a height of a fluid-sensor interface comprising an electrical double layer in the fluid enabling detection and characterization of the analyte.

However, such device requires a suitable semiconducting material to allow signal transduction and modulation between the electrodes and the analyte. The device may also require a selective linker chemistry to conjugate with a specific analyte and thus, the device is restricted to a particular analyte for performing characterisation. Therefore, there is a need for a device and method that addresses the abovementioned drawbacks.

### SUMMARY OF INVENTION

In one aspect of the present invention, a biosensor (100, 200) for detecting and characterising a biological material is provided. The biosensor (100, 200) comprises at least two electrodes (110, 210), a supply module configured to supply a fixed voltage over two of the at least two electrodes (110, 210), a frequency controller (130, 230) is configured to apply a signal at a driving frequency over two of the at least two electrodes (110, 210) and a measurement module (140, 240) is configured to measure at least one corresponding electrical parameter of the biological material,

Preferably, the at least two electrodes (110, 210) are metal layers fabricated spaced apart on a substrate (120, 220).

Preferably, the supply module is connected to a second electrode (212) and a third electrode (213), the frequency controller (230) is connected to a first electrode (211) and the third electrode (213). The measurement module (240) is connected to the second electrode (212) and the third electrode (213).

In another aspect of the present invention, a method for characterising a biological material is provided. The method is characterised by the steps of depositing a trace amount of the biological material onto at least two electrodes (110, 210); inducing a fixed voltage and a varying driving frequency to the biological material over two of the at least two electrodes (110, 210); measuring at least one corresponding electrical parameter of the biological material across the electrodes (110, 210) at the varying driving frequency; determining at least one peak or valley from the at least one corresponding electrical parameter at the varying driving frequency, wherein the at least one peak refers to a significant increase in the at least one corresponding electrical parameter at the varying driving frequency and the at least one valley refers to a significant drop in the at least one corresponding electrical parameter at the varying driving frequency; and defining characteristics of the at least one peak or valley as an electronic characteristic specific to the biological material.

Preferably, the fixed voltage is induced over a second electrode (212) and a third electrode (213), the varying driving frequency is applied over the first electrode (211) and the third electrode (213), and the at least one corresponding electrical parameter is measured across a second electrode (212) and the third electrode (213).

In yet another aspect of the present invention, a method for detecting at least one unknown biological material in a sample is provided. The method is characterised by the steps of depositing a trace amount of the sample onto at least two electrodes (110, 210); inducing a fixed voltage and a varying driving frequency to the sample over two of the at least two electrodes (110, 210); measuring the at least one corresponding electrical parameter of the at least one unknown biological material across the at least two electrodes (110, 210) at the varying driving frequency; determining at least one peak or valley from the at least one corresponding electrical parameter at the varying driving frequency, wherein the at least one peak refers to a significant increase in the at least one corresponding electrical parameter at the varying driving frequency and the at least one valley refers to a significant drop in the at least one corresponding electrical parameter at the varying driving frequency; and comparing characteristics of the at least one peak or valley of the unknown biological material with a database having a list of known biological materials and its respective electronic characteristics to identify the at least one unknown biological material.

Preferably, the fixed voltage is induced over a second electrode (212) and a third electrode (213), the varying driving frequency is applied over the first electrode (211) and the third electrode (213), and the at least one corresponding electrical parameter is measured across a second electrode (212) and the third electrode (213).

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 shows a first biosensor (100) for detecting and characterizing a biological material according to a first embodiment of the present invention.

FIG. 2 shows a second biosensor (200) for detecting and characterizing a biological material according to a second embodiment of the present invention.

FIGS. 3(a-b) show cross-sectional views of electrodes (110. 210) of the first and second biosensors (100, 200) deposited with a sample solution (10).

FIGS. 4(a-b) show exemplary graphs of measured current against driving frequency obtained by using the first and second biosensors (100, 200).

FIG. 5 shows an experimental setup of the first biosensor (100) of FIG. 1.

FIG. 6 shows a graph of measured current against a varying driving frequency for an algae specimen of Cyanobacteria sp.

FIGS. 7(a-b) show a first printed circuit board (120a) for the first biosensor (100) of FIG. 1 and a second printed circuit board (220a) for the second biosensor (200) of FIG. 2.

FIGS. 8(a-c) show graphs of measured impedance against a varying driving frequency for a synthetic micro RNA, a synthetic messenger RNA and a combination thereof.

### DESCRIPTION OF THE PREFERRED EMBODIMENT

A preferred embodiment of the present invention will be described herein below with reference to the accompanying drawings. In the following description, well-known functions or constructions are not described in detail since they would obscure the description with unnecessary detail.

The present invention relates to a biosensor for detecting and characterising a biological material in a sample solution. The term “biological material” used herein may refer to biomolecules such as but not restricted to protein, DNA, RNA; microorganism such as virus, bacteria and fungus; or the like. The biosensor detects and characterises the biological material based on its electronic properties. As the biosensor is based on electronic sensing, the biosensor may be applied as a test-kit for rapid detection of the biological material. The biosensor comprises at least two electrodes disposed on a substrate, a supply module, a frequency controller and a measurement module. The supply module, frequency controller and measurement module are connected to the at least two electrodes. The biosensor is preferably housed in a medical-grade stainless steel case or any equivalent material.

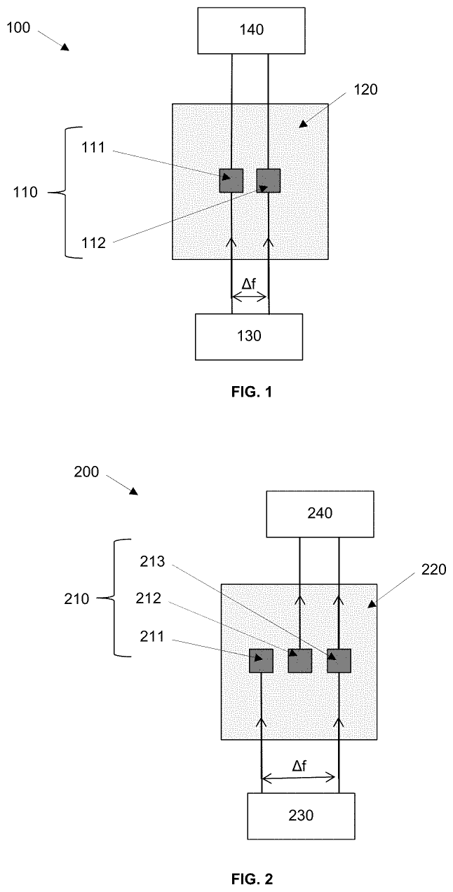

FIG. 1 illustrates a first biosensor (100) for detecting and characterising a biological material according to a first embodiment of the present invention. The first biosensor (100) includes two electrodes (110) disposed on a substrate (120), a supply module (not shown), a frequency controller (130), and a measurement module (140). Preferably, the two electrodes (110) are two metal layers fabricated side-by-side on an FR4 substrate (120), wherein the two metal layers are suitably made out of gold, aluminium or any other conducting material.

The supply module (not shown) is configured to supply a fixed voltage over the two electrodes (110) while the frequency controller (130) is configured to apply a signal at a driving frequency. The measurement module (140) is configured to measure at least one corresponding electrical parameter of the biological material, wherein the electronic parameter may include, but not limited to current, impedance, resistance, and capacitance. Although the supply module, the frequency controller (140), and the measurement module (130) as multiple independent units, it would be apparent by a person skilled in the art that the supply module, the frequency controller (130), and the measurement module (140) may be a single integrated unit.

FIG. 2 illustrates a second biosensor (200) for detecting and characterising a biological material according to a second embodiment of the present invention. The second biosensor (200) includes three electrodes (210) disposed on a substrate (220), and a source measurement unit (230). Preferably, the three electrodes (210) are three metal layers fabricated spaced apart on an FR4 substrate (220), wherein the three metal layers are suitably made out of gold, platinum or any other conducting material.

The supply module (not shown) is configured to supply a fixed voltage over two of three electrodes (210). In particular, the supply module is connected to a second electrode (212) and a third electrode (213). The frequency controller (230) is configured to apply a signal at a driving frequency. The frequency controller (230) is connected to a first electrode (211) and the third electrode (213). The measurement module (240) is configured to measure at least one corresponding electrical parameter of the biological material, wherein the electrical parameter may include, but not limited to current, impedance, resistance, and capacitance. The measurement module (240) is connected to the second electrode (212) and the third electrode (213). Although the supply module, the frequency controller (240), and the measurement module (230) as multiple independent units, it would be apparent by a person skilled in the art that the supply module, the frequency controller (230), and the measurement module (240) may be a single integrated unit.

A method for characterising a biological material in a sample solution is provided hereinbelow. The method is based on a principle that an intrinsic frequency of the biological material is responsive to a similar driving frequency and thereby, resulting in a resonance frequency and/or other natural phenomena that can be used to characterise a specific biological material. Such intrinsic frequency is unique to a particular type of biological material.

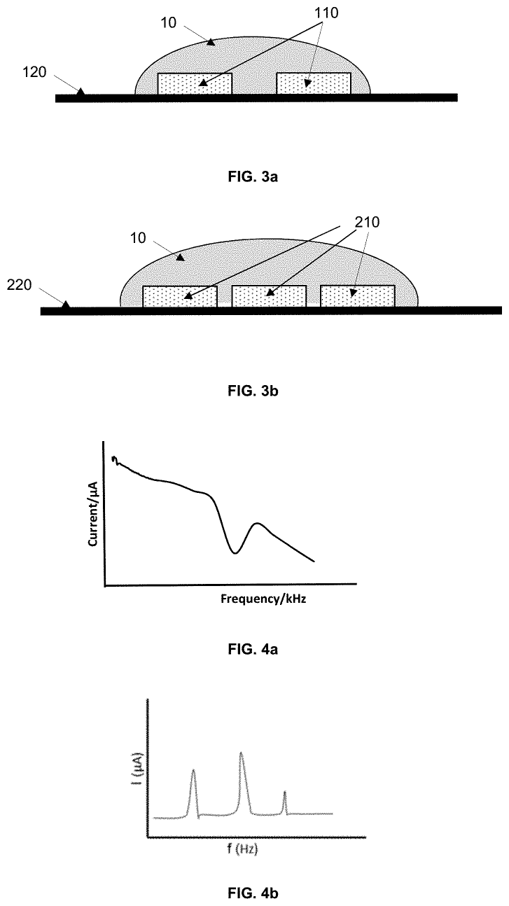

Initially, a trace amount of the sample solution is deposited onto the at least two electrodes (110, 210). The sample solution may be obtained from any liquid material such as sweat, blood, saliva, water and etc. The trace amount of sample solution (10) should be sufficient to cover a portion of all electrodes (110, 210) as shown in FIGS. 3(a-b).

Thereon, the supply module induces a fixed voltage or potential difference over two of the at least two electrodes (110, 210) while the frequency controller (130, 230) provides a signal at a driving frequency to the sample solution over two of the at least two electrodes (110, 210). For the first biosensor (100), the fixed potential difference is supplied over the two electrodes (110). For the second biosensor (200), the fixed potential difference is supplied over the second and third electrodes (212, 213), and the driving frequency is provided over the first and third electrodes (212, 213). The driving frequency is provided at a varying frequency.

The measurement module (140, 240) measures at least one corresponding electrical parameter of the biological material across the electrodes (110, 210) at the varying driving frequency, wherein the electrical parameter may include, but not limited to current, impedance, resistance, and capacitance. For the first biosensor (100), the at least one corresponding electrical parameter is measured across the first and second electrodes (111, 112) and as for the second biosensor (200), the at least one corresponding electrical parameter is measured across the second and third electrodes (212, 213).

Thereon, one or more peaks or valleys are determined from the at least one corresponding electrical parameter measured at the varying frequency, wherein the measured electronic property can be plotted by a graph of current against frequency, impedance against frequency, resistance against frequency, or capacitance against frequency. The one or more peaks or valleys reflect the resonance frequency produced as the intrinsic frequency of the biological material matches the driving frequency. In one example, FIG. 4a shows a graph of the measured current against the driving frequency whereby the resonance frequency is reflected by a significant drop or valley of the measured current. In another example, FIG. 4b shows an exemplary graph of the measured current against the driving frequency whereby the resonance frequency is reflected by three significant peaks of the measured current indicating the existence of three different types of biomolecules in the sample within the range of the driving frequency.

The characteristic of the peaks or valleys is determined to be an electronic characteristic specific to the biological material in the sample solution. The characteristic of the peaks or valleys may include frequency of the peaks or valleys, and the measured electronic property at the peaks or valleys.

A method for detecting at least one unknown biological material in a sample solution is provided hereinbelow. The method is based on a principle that an intrinsic frequency of the biological material is responsive to a similar driving frequency and thereby, resulting in a resonance frequency that can be used to detect and characterise the presence of at least one specific biological material in the sample solution. Such intrinsic frequency is unique to a particular type of biological material.

Initially, a trace amount of the sample solution is deposited onto the at least two electrodes (110, 210). The sample solution may be obtained from any liquid material such as sweat, blood, saliva, water and etc. The trace amount of sample solution should be sufficient to cover a portion of all electrodes (110, 210) as shown in FIGS. 3(a-b).

Thereon, the supply module induces a fixed voltage or potential difference over two of the at least two electrodes (110, 210) while the frequency controller (130, 230) provides a signal at a driving frequency to the sample solution over two of the at least two electrodes (110, 210). For the first biosensor (100), the fixed potential difference is supplied over the two electrodes (110). For the second biosensor (200), the fixed potential difference is supplied over the second and third electrodes (212, 213), and the driving frequency is provided over the first and third electrodes (212, 213). The driving frequency is provided at various frequencies, preferably in ascending order so as to detect all possible biological materials in the sample solution.

The measurement module (140, 240) measures at least one corresponding electrical parameter of the at least one unknown biological material across the electrodes (110, 210) at the varying driving frequency. For the first biosensor (100), the at least one corresponding electrical parameter is measured across the first and second electrodes (111, 112) and as for the second biosensor (200), the at least one corresponding electrical parameter is measured across the second and third electrodes (212, 213). The at least one corresponding electrical parameter may include, but not limited to current, impedance, resistance, and capacitance.

Thereon, one or more peaks or valleys are determined from the at least one corresponding electrical parameter measured at the varying driving frequency. The measured electrical parameter can be plotted by a graph of current against frequency, impedance against frequency, resistance against frequency, or capacitance against frequency. It would be appreciated by a person skilled in the art that the measured electrical parameter at the varying driving frequency may be subjected to any signal processing algorithms such as Fast Fourier Transform in order to reduce noise and/or to clearly identify the peaks or valleys within the signal or graph of the measured electrical parameter.

The characteristic of the peaks or valleys is determined to be the electronic characteristic of the at least one unknown biological material in the sample solution. The characteristic of the peaks or valleys may include frequency of the peaks or valleys, and the measured electronic property at the peaks or valleys.

The electronic characteristic of the at least one unknown biological material is then compared with a database having a list of known biological materials and its respective electronic characteristics. If the electronic characteristic of the at least one unknown biological material matches one of the electronic characteristics in the database, the at least one unknown biological material is detected and identified as the particular biological material having the matched electronic characteristics stored in the database. Once the database is established for the biological materials of interest, for example, a virus, only the selected corresponding range of frequencies are then applied to make a positive detection of the specific virus of interest

Hereinafter, the present invention is further illustrated by the following examples. The examples used herein are intended merely to facilitate an understanding of ways in which the embodiments herein may be practised and to further enable those of skill in the art to practise the embodiments herein.

### Example 1

### Experimental Setup of the Biosensor

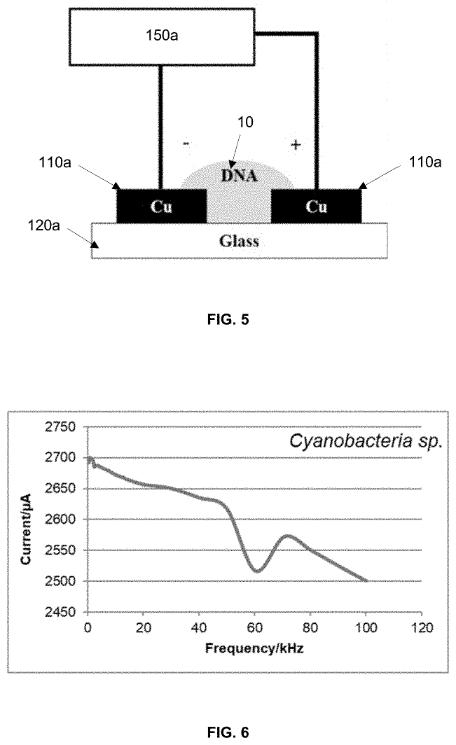

FIG. 5 shows a block diagram of an experimental setup of the first biosensor. Two copper strips (110a) were used as the electrodes, wherein the copper strips (110a) were disposed on a glass substrate (120a) side-by-side with a 0.5 mm gap in between the copper strips (110a). One of the copper strips (110a) was connected to a positive terminal of a source measurement unit (150a) while another copper strip (110a) was connected to a negative terminal of the source measurement unit (150a). The source measurement unit (150a) was used to supply a fixed potential difference, provide a varying driving frequency, and measure current produced over the electrodes (110a) as the driving frequency increases. Thus, the source measurement unit (150a) was used as the supply module, the frequency controller (130), and the measurement module (140).

### Detection and Characterisation of Algae Specimen

A portion of an algae test subject was taken from Cyanobacteria sp. to prepare as an algae specimen. Thereon, 10 μL of the algae specimen (10) was deposited onto the copper strips (110a) whereby a portion of both copper strips (110a) was in contact with the deposited algae specimen (10). A fixed potential difference of approximately 1.5 V and a varying driving frequency were applied to the algae specimen (10) through the copper strips (110a) by the source measurement unit (150a). Concurrently, the current produced over the electrodes (110a) was measured as the driving frequency increases.

As a result, a graph of the measured current against the driving frequency of the algae specimen was plotted as shown in FIG. 6. A significant current drop was observed at 60 kHz driving frequency. Hence, the electronic characteristic of Cyanobacteria sp is defined by the characteristic of the significant current drop at the driving frequency of 60 kHz.

### Example 2

### Fabrication of the Electrodes for the Biosensor

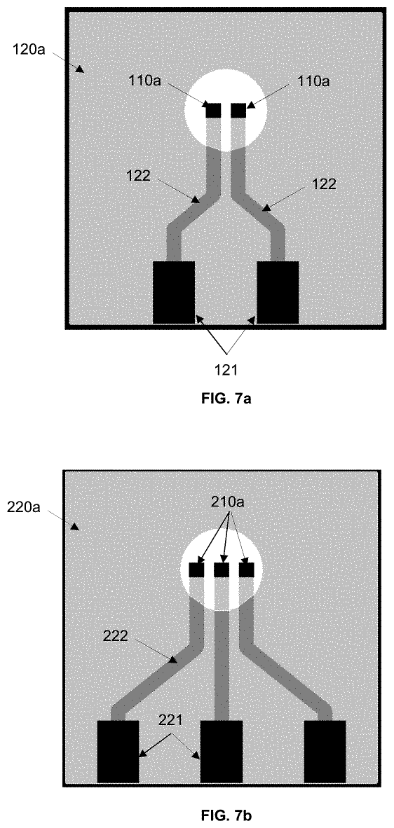

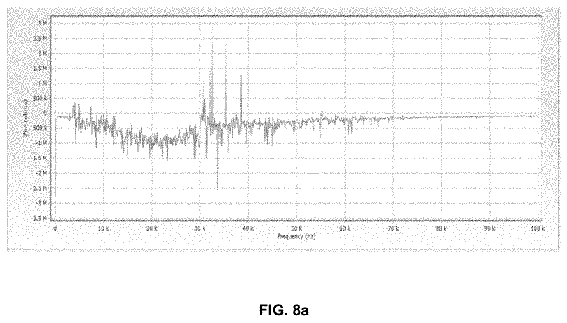

A first printed circuit board or PCB (120a) was fabricated for the first biosensor. The design of the first PCB (120a) is as shown in FIG. 7a. The first PCB (120a) was a single-sided FR4 1.6 mm designed with two electrodes (110a) and two connecting pads (121), wherein each electrode (110a) was connected to one of the connecting pads (121) by a copper track (122) with a thickness of approximately 36 μm. The electrodes (110a) and connecting pads (121) were electroplated nickel-gold plates having a nickel thickness layer of approximately 4 to 5 μm and a nickel thickness layer of approximately 0.049 to 0.052 μm. The copper tracks (122) were covered by epoxy solder mask to allow only the electrodes (110a) and the connecting pads (121) being exposed. The first PCB (120a) was pre-treated by immersing in acetone for 60 s, rinsing using deionized water, immersing in isopropanol for 60 s, rinsing using deionized water, and drying using Nitrogen gas.

### Example 3

### Fabrication of the Electrodes for the Biosensor

A second printed circuit board or PCB (220a) was fabricated for the second biosensor. The design of the PCB (220a) is as shown in FIG. 7b. The second PCB (220a) was a single-sided FR4 1.6 mm designed with three electrodes (210a) and three connecting pads (221), wherein each electrode (210a) was connected to one of the connecting pads (221) by a copper track (222) with a thickness of approximately 36 μm. The electrodes (210a) and connecting pads (221) were electroplated nickel-gold plates having a nickel thickness layer of approximately 4 to 5 μm and a nickel thickness layer of approximately 0.049 to 0.052 μm. The copper tracks (222) were covered by epoxy solder mask to allow only the electrodes (210a) and the connecting pads (221) being exposed. Prior to the experiments, the second PCB (220a) was pre-treated by sonicating in acetone, rinsing using deionized water, sonicating in ethanol, rinsing using deionized water, and drying using Nitrogen gas.

### Experimental Setup of the Biosensor

The second PCB (220a) was connected to a potentiostat with built-in frequency controller via the connecting pads (221). The potentiostat was used to supply a fixed potential difference and provide a varying driving frequency to the first sample through the electrodes (210a). The potentiostat was also used to measure impedance produced over the electrodes (210a) as the driving frequency increases. Thus, the potentiostat was used as the supply module, the frequency controller (230), and the measurement module (240).

### Detection and Characterisation of Synthetic RNA

A first sample of synthetic micro RNA was obtained. 10 μL of the first sample was deposited onto the electrodes (210a) of the second PCB (220a). A fixed potential difference of approximately 0.5 V and a varying driving frequency were applied to the sample through the electrodes (210a) by the potentiostat. Concurrently, impedance produced over the electrodes (210a) was measured as the driving frequency increases.

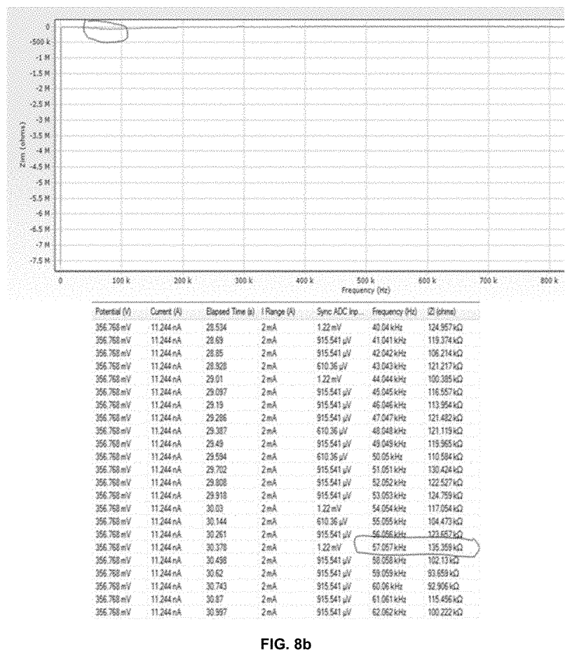

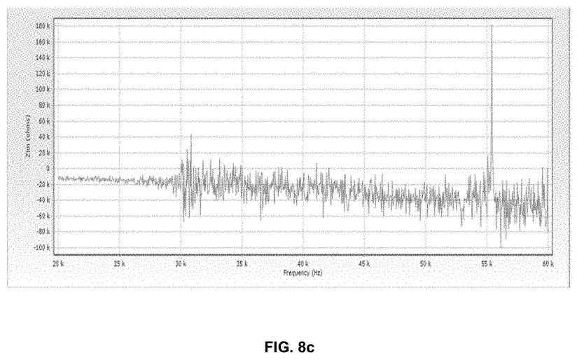

As a result, a graph of the measured impedance against the driving frequency was obtained as shown in FIG. 8a. Significant impedance peaks were observed at the driving frequency range of 30 to 40 kHz. Hence, the electronic characteristic of the synthetic micro RNA is defined by the characteristic of the significant impedance peaks within the driving frequency range of 30 to 40 kHz and may be defined further with further investigations.

### Example 4

### Experimental Setup of the Biosensor

The second PCB (220a) as shown in FIG. 7b was used in this experimental setup whereby the second PCB (220a) was connected to a potentiostat with built-in frequency controller via the connecting pads (221). The potentiostat was used to supply a fixed potential difference and provide a varying driving frequency to the first sample through the electrodes (210a). The potentiostat was also used to measure impedance produced over the electrodes (210a) as the driving frequency increases. Thus, the potentiostat was used as the supply module, the frequency controller (230), and the measurement module (240).

### Detection and Characterisation of Synthetic RNA

A second sample of synthetic messenger RNA was obtained. 10 μL of the second sample was deposited onto the electrodes (210a) of the second PCB (220a). A fixed potential difference of approximately 0.5 V and a varying driving frequency were applied to the sample through the electrodes (210a) by the potentiostat. Concurrently, impedance produced over the electrodes (210a) was measured as the driving frequency increases.

As a result, a graph of the measured impedance against the driving frequency was obtained as shown in FIG. 8b. A significant impedance peak of approximately 135.36 kΩ was observed at the driving frequency of approximately 57 kHz. Hence, the electronic characteristic of the synthetic messenger RNA is defined by the characteristic of the significant impedance peak at the driving frequency range of approximately 57 kHz.

### Example 5

### Experimental Setup of the Biosensor

The second PCB (220a) as shown in FIG. 7b was used in this experimental setup whereby the PCB (220a) was connected to a potentiostat with built-in frequency controller via the connecting pads (221). The potentiostat was used to supply a fixed potential difference and provide a varying driving frequency to the first sample through the electrodes (210a). The potentiostat was also used to measure impedance produced over the electrodes (210a) as the driving frequency increases. Thus, the potentiostat was used as the supply module, the frequency controller (230), and the measurement module (240).

### Detection and Characterisation of Synthetic RNA

A third sample was prepared by mixing the synthetic messenger RNA and the synthetic micro RNA. 10 μL of the third sample was deposited onto the electrodes (210a) of the second PCB (220a). A fixed potential difference of approximately 0.5 V and a varying driving frequency were applied to the sample through the electrodes (210a) by the potentiostat. Concurrently, impedance produced over the electrodes (210a) was measured as the driving frequency increases.

As a result, a graph of the measured impedance against the driving frequency was obtained as shown in FIG. 8b. A significant impedance peak was observed at the driving frequency of approximately 55 kHz and significant peaks were also observed at the driving frequency range of 30 to 40 kHz. The impedance peaks were consistent with the impedance peaks as observed in Example 3 and 4. Hence, the biosensor (100, 200) would be able to perform multiple detections within a sample whereby in this case the biosensor was able to detect both synthetic micro RNA and messenger RNA within one sample.

While embodiments of the invention have been illustrated and described, it is not intended that these embodiments illustrate and describe all possible forms of the invention. Rather, the words used in the specifications are words of description rather than limitation and various changes may be made without departing from the scope of the invention.

1. A biosensor for detecting and characterising a biological material comprising at least two electrodes, characterised in that the biosensor further includes:
a supply module configured to supply a fixed voltage over two of the at least two electrodes;
a frequency controller is configured to apply a signal at a driving frequency over two of the at least two electrodes; and
a measurement module is configured to measure at least one corresponding electrical parameter of the biological material.

2. The biosensor as claimed in claim 1, wherein the at least two electrodes are metal layers fabricated spaced apart on a substrate.

3. The biosensor as claimed in claim 1, wherein the supply module is connected to a second electrode and a third electrode the frequency controller is connected to a first electrode and the third electrode and the measurement module is connected to the second electrode and the third electrode.

4. A method for characterising a biological material is characterised by the steps of:
a) depositing a trace amount of the biological material onto at least two electrodes;
b) inducing a fixed voltage and a varying driving frequency to the biological material over two of the at least two electrodes;
c) measuring at least one corresponding electrical parameter of the biological material across the electrodes at the varying driving frequency;
d) determining at least one peak or valley from the at least one corresponding electrical parameter at the varying driving frequency, wherein the at least one peak refers to a significant increase in the at least one corresponding electrical parameter at the varying driving frequency and the at least one valley refers to a significant drop in the at least one corresponding electrical parameter at the varying driving frequency; and
e) defining characteristics of the at least one peak or valley as an electronic characteristic specific to the biological material.

5. The method as claimed in claim 4, wherein the fixed voltage is induced over a second electrode and a third electrode, the varying driving frequency is applied over the first electrode and the third electrode and the at least one corresponding electrical parameter is measured across a second electrode and the third electrode.

6. A method for detecting at least one unknown biological material in a sample is characterised by the steps of:
a) depositing a trace amount of the sample onto at least two electrodes;
b) inducing a fixed voltage and a varying driving frequency to the sample over two of the at least two electrodes;
c) measuring the at least one corresponding electrical parameter of the at least one unknown biological material across the at least two electrodes at the varying driving frequency;
d) determining at least one peak or valley from the at least one corresponding electrical parameter at the varying driving frequency, wherein the at least one peak refers to a significant increase in the at least one corresponding electrical parameter at the varying driving frequency and the at least one valley refers to a significant drop in the at least one corresponding electrical parameter at the varying driving frequency; and
e) comparing characteristics of the at least one peak or valley of the at least one unknown biological material with a database having a list of known biological materials and its respective electronic characteristics to identify the at least one unknown biological material.

7. The method as claimed in claim 6, wherein the fixed voltage is induced over a second electrode and a third electrode, the varying driving frequency is applied over the first electrode and the third electrode, and the at least one corresponding electrical parameter is measured across a second electrode and the third electrode.
